(19) United States
(12) Patent Application Publication
(10) Pub. No.: US 2015/0038493 A1
(43) Pub. Date: Feb. 5, 2015

(54) COMPOUNDS THAT TRAP ALPHA-OXOALDEHYDES AND ALPHA-BETA-UNSATURATED ALDEHYDES, META-COMPOUNDS CONTAINING SUCH COMPOUNDS, AND USE OF SAID COMPOUNDS IN TREATING ILLNESSES RELATED TO THE ACCUMULATION OF AGES AND ALES

(71) Applicant: PHARMAMENS, Paris (FR)

(72) Inventors: Guy Potier, Paris (FR); André Nobumichi Sasaki, France (FR); Nicolas Audic, Amiens (FR)

(21) Appl. No.: 14/350,126

(22) PCT Filed: Oct. 5, 2012
(86) PCT No.: PCT/FR2012/052268

§ 371 Date: Apr. 7, 2014

(30) Foreign Application Priority Data
Oct. 7, 2011 (FR) 1159061

(51) Int. Cl. C07D 295/16; C07D 211/32; C07D 223/04; A23L 1/30; C07D 215/06; A61K 8/49; A61Q 19/08; C07D 207/09; C07D 243/08

(52) U.S. Cl. CPC C07D 295/16; C07D 207/09; C07D 211/32; C07D 223/04; C07D 243/08; C07D 215/06; A61K 8/494; A61K 8/4926; A61K 8/4913; A61Q 19/08; A23L 1/30; A23V 2002/00
USPC 514/217.11; 548/540; 514/423; 546/245; 514/315; 540/607; 544/168; 514/237.5; 544/58.4; 514/227.5; 544/386; 514/255.01; 544/391; 544/387; 540/575; 514/218; 546/179; 544/363; 514/253.06

(57) ABSTRACT

The present invention relates to a series of molecules derived from 2,3-diaminopropionic acid (DAP), comprising or not comprising an 8-hydroxyquinoline (8-HQ) motif, and to the use of said molecules for trapping an alpha-oxoaldehyde resulting particularly from the degradation of the glucose or for trapping an alpha-beta-unsaturated aldehyde, resulting particularly from the oxidative degradation of fatty acids. These molecules can have a further application in the fields of cosmetics, food processing, and pharmaceuticals.

### TECHNICAL FIELD

The present invention relates to a series of molecules derived from 2,3-diaminopropionic acid (Dap), optionally comprising an 8-hydroxyquinoline (8-HQ) unit, and to the use thereof for scavenging an alpha-oxoaldehyde in particular resulting from glucose degradation and/or for scavenging an alpha, beta-unsaturated aldehyde, in particular resulting from the oxidative degradation of fatty acids. These molecules can also have an application in the cosmetics, agri-food and pharmaceutical fields.

Indeed, these novel compounds have the ability to effectively scavenge α-oxoaldehydes resulting from glucose degradation (for example, glyoxal, methylglyoxal, 3-deoxyglucosone) and/or α,β-unsaturated aldehydes resulting from the oxidative degradation of certain fatty acids (for example, acrolein, malondialdehyde, 4-hydroxynonenal). These aldehydes are partly responsible for irreversible modifications on proteins known under the generic terms AGEs (Advanced Glycation End-products) and ALEs (Advanced Lipid peroxidation End-products). The accumulation of these modifications is, inter alia, closely linked to the development of vascular complications in diabetics, such as atherosclerosis, retinopathy or nephropathy, and of certain neurodegenerative pathological conditions such as Alzheimer's disease.

The therapeutic use of the novel molecules described hereinafter is therefore of particularly advantageous interest for preventing the occurrence of these pathological conditions or even slowing down or treating the development thereof. However, their use in the cosmetics and food fields is also of interest, quite particularly in the treatment or prevention of skin aging.

### TECHNOLOGICAL BACKGROUND

The reaction for non-enzymatic condensation between sugars and the amino groups of proteins, i.e. a Maillard reaction, results in the formation of “Advanced Glycation End-products”, or AGEs, on proteins. The in vivo occurrence of these irreversible modifications is a lengthy and complex process which has been shown to involve not only sugars, such as glucose, but also some of their degradation products and metabolites, of α-oxoaldehyde type, for instance methylglyoxal (abbreviated as MGO), glyoxal (abbreviated as GO) or 3-deoxyglucosone (abbreviated as 3-DG).

The accumulation of AGEs has two major biological consequences. Firstly, protein crosslinking: this phenomenon is mainly observed on proteins with a long lifetime (collagen, proteins of the crystalline lens, fibronectin, albumin, hemoglobin, etc.) and plays a predominant role in normal aging (loss of physical flexibility of tissues, skin pigmentation) and the occurrence of specific pathological conditions of elderly individuals (cataracts, rheumatic disorders). Secondly, the generation of oxidative stress at the cellular level results in the occurrence of inflammatory and thrombogenic reactions via interaction between AGEs and certain specific receptors (RAGEs). It has thus been possible to demonstrate several intracellular event cascades, initiated by AGE/RAGE interactions, and to directly link them to the development of atherosclerosis and of various microvascular complications (nephropathy, cardiovascular disorders, retinopathy, neuropathy). Persistent hyperglycemic states cause considerable increases in the production of α-oxoaldehydes and in the formation of AGEs; consequently, diabetic individuals are particularly affected by the pathological states mentioned above. The accumulation of AGEs is also involved in the development of certain neurodegenerative diseases such as Alzheimer's disease.

The inhibition of AGE formation and more precisely the in vivo scavenging of the α-oxoaldehydes generated from glucose therefore constitute a therapeutic approach of great interest with regard to the prevention and treatment of the diseases mentioned above. Several compounds have been developed for this purpose since the middle of the 1990s.

Aminoguanidine is one of the most widely studied molecules. In addition to its ability to effectively scavenge MGO and GO, aminoguanidine is a good nitric oxide synthase (abbreviated as NOS) inhibitor and has shown itself to be capable of stopping the development of retinopathy and also of slowing down nephropathic complications in diabetic rats. The development of this molecule has, however, been suspended owing to adverse hepatic and gastrointestinal side effects which occurred during a clinical trial relating to the prevention of the progression of diabetic nephropathy.

Pyridoxamine is also an excellent α-oxoaldehyde scavenger compound which has been found to be capable of reducing the pathological complications usually observed in diabetic rats.

Oxidative stress states, resulting from an imbalance between production of reactive oxygen species (free radicals) and antioxidant cell defenses, are one of the major consequences of diabetes and are, inter alia, responsible for the lipid peroxidation phenomenon. This oxidizing process results in the fragmentation of polyunsaturated fatty acids and in the formation of alpha, beta-unsaturated aldehydes, such as acrolein (abbreviated as ACR), malondialdehyde (abbreviated as MDA) or 4-hydroxy-2-nonenal (abbreviated as 4-HNE), and also of α-oxoaldehydes previously mentioned (abbreviated as MGO, GO). Alpha, beta-unsaturated aldehydes are highly toxic compounds capable of reacting with proteins so as to result in the formation of adducts known under the generic term ALEs (Advanced Lipid peroxidation End-products). Just like AGEs, ALEs induce cell dysfunctions and protein crosslinking phenomena.

Among the alpha, beta-unsaturated aldehydes, acrolein is the compound which exhibits the greatest reactivity toward cysteine, histidine and lysine residues of proteins. The accumulation of the acrolein-derived ALE N-(3-formyl-3,4-dehydropiperidino)lysine (abbreviated as FDP) is thus strongly suspected of contributing to the formation of the abnormalities on Müller glial cells encountered in cases of diabetic retinopathy. High levels of acrolein adducts have also been demonstrated in some neuronal proteins in patients suffering from Alzheimer's disease. Likewise, 4-HNE exhibits an entire range of harmful biological effects, ranging from impairment of genetic expression up to cell apoptosis. Exposure to 4-HNE is implicated in the etiology of numerous diseases associated with oxidative stress, such as atherosclerosis, hepatic ischemia-reperfusion lesions, Alzheimer's disease and Parkinson's disease. Finally, malondialdehyde is known to react with the lysine residues of proteins so as to form dihydropyridine (DHP) derivatives, the presence of which results in phenomena of UV-sensitization of the skin, which contribute to accelerated aging, or even to cancer, of the skin. MDA levels are notably higher in diabetic patients and cause, through crosslinking, deleterious stiffening of collagen in the cardiovascular system. The mutagenic nature of MDA owing to its reactivity toward DNA may also be noted.

One of the therapeutic strategies most widely used for countering the harmful effects of alpha, beta-unsaturated aldehydes consists in using against them nucleophilic molecules capable of diverting them from their biological targets, namely proteins or DNA.

It is therefore of great interest to have novel molecules, devoid of side effects, which make it possible to act on the modifications of ALEs and AGEs by preventing their accumulation responsible for numerous pathological conditions.

### DESCRIPTION OF THE INVENTION

In the context of the present invention:
the term “alkyl group” is intended to mean a linear, branched or cyclic, saturated aliphatic group optionally substituted with a linear, branched or cyclic, saturated alkyl group. By way of examples, mention may be made of methyl, ethyl, propyl, isopropyl, butyl, isobutyl, tert-butyl, cyclopropyl, cyclobutyl, cyclopentyl, cyclohexyl, methylcyclopropyl and cyclopropyl-methyl groups;
the term “aryl group” is intended to mean a monocyclic or bicyclic aromatic group comprising from 6 to 10 atoms. By way of examples of aryl groups, mention may be made of phenyl, pyridyl and pyrimidyl;
the term “aralkyl group” is intended to mean a linear or branched, saturated aliphatic group substituted with at least one monocyclic or bicyclic aromatic group comprising from 6 to 10 atoms. By way of example of aralkyl groups, mention may be made of benzyl.
   A subject of the present invention is a compound of formula (I) below, in free form or in the form of salts of at least one inorganic or organic acid,

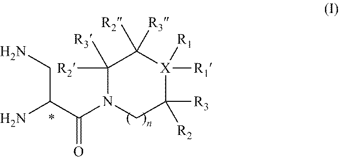

in which:
R2, R2′ and R2″ are selected from a hydrogen atom, an alkyl group, an aralkyl group and an aryl group;
R3, R3′ and R3″ are selected from a hydrogen atom, an alkyl group, an aralkyl group and an aryl group;
n is an integer equal to 0, 1 or 2;
X is an atom selected from O, S, C and N atoms;
   provided that, when X is an O or S atom, then R1 and R1′ are absent; and
   provided that, when X is a C atom, then
      (i) R1 and R1′, independently of one another, are selected from a hydrogen atom, an alkyl group and an aryl group, or
      (ii) R1 is selected from a hydrogen atom, an alkyl group and an aryl group, and R1′ is connected to R2, R2′, R2″, R3, R3′ or R3″ with a methylene (—CH₂—), oxo (—O—), thio (—S—) or imino (—NH—) moiety so as to form a ring, or
      (iii) R1 and R1′, independently of one another, are connected to R2, R2′, R2″, R3, R3′ or R3″ with a methylene (—CH₂—), oxo (—O—), thio (—S—) or imino (—NH—) moiety so as to form a ring, or
   provided that, when X is an N atom, then (i) R1′ is absent and (ii) R1 is selected from a hydrogen atom, an alkyl group, a hydroxyalkyl group, an aralkyl group, an aryl group, and a moiety having the structure

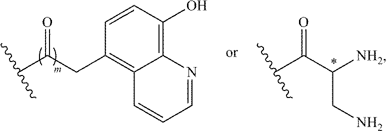

with m an integer selected from 0 and 1, or R1 is connected to R2, R2′, R2″, R3, R3′ or R3″ with a methylene (—CH₂—), oxo (—O—), thio (—S—) or imino (—NH—) moiety so as to form a ring,
   and in which:
“*” means that the corresponding asymmetric carbon atom is of R or S stereochemistry;
   with the proviso that the following compounds are excluded:
n is 0, X is a C atom and R1, R1′, R2, R2′, R2″, R3, R3′ and R3″ are hydrogen atoms;
n is 1 and X is an O or S atom;
n is 1, X is a C atom and R1 and R1′ are hydrogen atoms;
n is 1, X is an N atom and R1 is a hydrogen atom;
n is 1, X is a C atom, R1, R2, R2′, R2″, R3, R3′ and R3″ are hydrogen atoms and R1′ is a methyl (—CH₃) group or a phenyl (—C₆H₅) group;
n is 1, X is an N atom, R2, R2′, R2″, R3, R3′ and R3″ are hydrogen atoms and R1 is a phenyl (C₆H₅) group or a benzyl (CH₂C₆H₅) group.
   The compounds of formula (I) comprising at least one asymmetric carbon atom can exist in the form of two enantiomers. These enantiomers, and also mixtures thereof, including racemic mixtures, are part of the invention.
   The compounds of formula (I) can exist in the form of bases or of addition salts with acids. Such addition salts are part of the invention. These salts can be prepared with pharmaceutically acceptable acids, but the salts of other acids, of use for example for purifying or isolating the compounds of formula (I), are also part of the invention.
   The compounds of formula (I) can also exist in the form of hydrates or of solvates, namely in the form of associations or combinations with one or more molecules of water or with a solvent. Such hydrates and solvates are also part of the invention.
   According to one embodiment, the compound (I) according to the invention may be of formula (Ia) below:

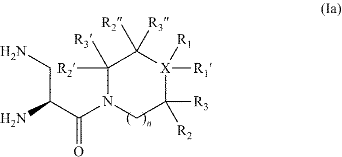

or of formula (Ib) below:

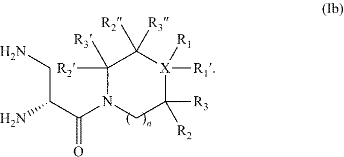

According to one embodiment, the compound (I) according to the invention of formula (Ia) below:

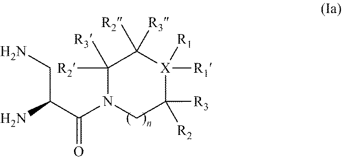

has an R1 group which may be selected from one of the following moieties:

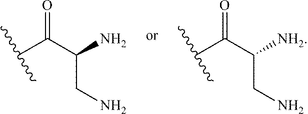 [SMILES: *C(=O)[C@@H](N)CN ; "or" ; *C(=O)[C@H](N)CN]

According to one embodiment, said compound of formula (Ib) below:

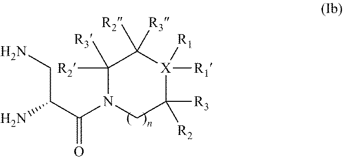

has an R1 group which may be selected from one of the following moieties:

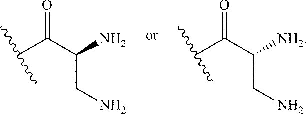 [SMILES: *C(=O)[C@@H](N)CN ; "or" ; *C(=O)[C@H](N)CN]

According to one embodiment, the compound according to the invention has a number n which is equal to 0 and an atom X which is a nitrogen atom.
According to one embodiment, the compound according to the invention has one or more group(s):
R2′, R2″, R3′ and R3″ which are other than a hydrogen atom; or
R2″ which is a hydrogen atom and R2′, R3′ and R3″ which are other than a hydrogen atom; or
R2′ which is a hydrogen atom and R2″, R3″ and R3′ which are other than a hydrogen atom; or
R2′ and R3′ which are hydrogen atoms and R2″ and R3″ which are other than a hydrogen atom; or
R2″ and R3″ which are hydrogen atoms and R2′ and R3′ which are other than a hydrogen atom; or
R2′ and R2″ which are hydrogen atoms and R3′ and R3″ which are other than a hydrogen atom; or
R2′, R2″, R3′ and R3″ which are hydrogen atoms.
   According to one embodiment, the organic or inorganic acid is selected from hydrochloric, hydrobromic, hydroiodic, sulfuric, phosphoric, tartaric, lactic, acetic, adipic, alginic, aspartic, benzoic, benzenesulfonic, bisulfic, butyric, citric, camphoric, camphorsulfonic, gluconic, dodecylsulfonic, ethanesulfonic, fumaric, glucoheptanoic, heptanoic, hexanoic, 2-hydroxyethanesulfonic, maleic, methanesulfonic, 2-naphthalenesulfonic, nicotinic, oxalic, palmoic, palmitic, pectinic, 3-phenylpropionic, picric, pivalic, propionic, succinic and undecanoic acids.
   According to one embodiment, the compound according to the invention is selected from:
4d: (2R)-2,3-diamino-1-(azepan-1-yl)propan-1-one;
4g: (2R)-2,3-diamino-1-(4-methylpiperazin-1-yl)propan-1-one;
4h: (2R)-2,3-diamino-1-(4-cyclohexylpiperazin-1-yl)propan-1-one;
4j: (2R)-2,3-diamino-1-[4-(2-hydroxyethyl)piperazin-1-yl]propan-1-one;
4k: (2R)-2,3-diamino-1-[4-(3-hydroxypropyl)piperazin-1-yl]propan-1-one;
4m: (2R)-2,3-diamino-1-(4-butylpiperazin-1-yl)propan-1-one;
6a: (2R,2′R)-1,1′-(piperazine-1,4-diyl)bis(2,3-diaminopropan-1-one);
6b: (2R,2′R)-1,1-(1,4-diazepane-1,4-diyl)bis(2,3-diaminopropan-1-one);
11: (2R)-2,3-diamino-1-{4-[(8-hydroxyquinolin-5-yl)methyl]piperazin-1-yl}propan-1-one; and
13: (2R)-2,3-diamino-1-{4-[2-(8-hydroxyquinolin-5-yl)acetyl]piperazin-1-yl}propan-1-one.
   The invention also relates to the use of a compound according to the invention for scavenging an alpha-oxoaldehyde or an alpha, beta-unsaturated aldehyde, or a compound according to the invention for use as an alpha-oxoaldehyde scavenger or scavenging agent or an alpha, beta-unsaturated aldehyde scavenger or scavenging agent.
   The alpha-oxoaldehyde may result from glucose degradation. The alpha-oxoaldehyde resulting from glucose degradation may be selected from glyoxal, methylglyoxal and 3-deoxyglucosone.
   The alpha, beta-unsaturated aldehyde may result from the oxidative degradation of a fatty acid. The alpha, beta-unsaturated aldehyde resulting from the oxidative degradation of a fatty acid may be selected from acrolein, malondialdehyde and 4-hydroxynonenal.
   The invention also relates to a compound according to the invention for use as a medicament.
   According to a first therapeutic use aspect, a subject of the invention is a compound according to the invention or a compound of formula (I)

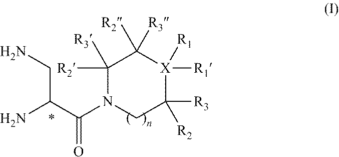

in which:
n is 0, X is a C atom and R1, R1′, R2, R2′, R2″, R3, R3′ and R3″ are hydrogen atoms;
n is 1, X is an O or S atom, R1 and R1′ are absent and R2, R3, R2′, R3′, R3″ and R2″ are selected from a hydrogen atom, an alkyl group and an aryl group;
n is 1, X is a C atom, R1 and R1′ are hydrogen atoms and R2, R3, R2′, R3′, R3″ and R2″ are selected from a hydrogen atom, an alkyl group and an aryl group;
n is 1, X is an N atom, R1 is a hydrogen atom and R2, R3, R2′, R3′, R3″ and R2″ are selected from a hydrogen atom, an alkyl group and an aryl group;
n is 1, X is a C atom, R1, R2, R2′, R2″, R3, R3′ and R3″ are hydrogen atoms and R1′ is a methyl (—CH₃) group or a phenyl (C₆H₅) group;
n is 1, X is an N atom, R2, R2′, R2″, R3, R3′ and R3″ are hydrogen atoms and R1 is a phenyl (—C₆H₅) group or a benzyl (—CH₂C₆H₅) group,
   for treating or preventing a neurodegenerative pathological condition. Said neurodegenerative pathological condition may be selected from Alzheimer's disease and Parkinson's disease.
   According to a second therapeutic use aspect, a subject of the invention is a compound according to the invention or a compound of formula (I)

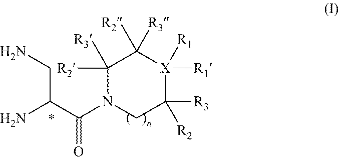

in which:
n is 0, X is a C atom and R1, R1′, R2, R2′, R2″, R3, R3′ and R3″ are hydrogen atoms;
n is 1, X is a C atom, R1, R2, R2′, R2″, R3, R3′ and R3″ are hydrogen atoms and R1′ is a methyl (CH₃) group or a phenyl (C₆H₅) group;
n is 1, X is an N atom, R2, R2′, R2″, R3, R3′ and R3″ are hydrogen atoms, R1′ is absent and R1 is a phenyl (C₆H₅) group or a benzyl (CH₂C₆H₅) group,
   for treating or preventing conditions associated with diabetes. Said conditions associated with diabetes may be selected from atherosclerosis, retinopathy, nephropathy, neuropathy, micro- and macroangiopathies, cataracts, amyloidosis, rheumatic disorders and varicose and arterial ulcers.
   According to a third therapeutic use aspect, a subject of the invention is a compound according to the invention or a compound of formula (I)

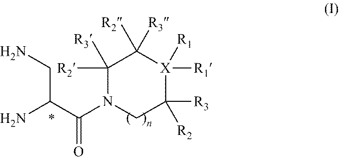

in which:
n is 0, X is a C atom and R1, R1′, R2, R2′, R2″, R3, R3′ and R3″ are hydrogen atoms;
n is 1, X is an O or S atom, R1 and R1′ are absent and R2, R3, R2′, R3′, R3″ and R2″ are selected from a hydrogen atom, an alkyl group and an aryl group;
n is 1, X is a C atom, R1 and R1′ are hydrogen atoms and R2, R3, R2′, R3′, R3″ and R2″ are selected from a hydrogen atom, an alkyl group and an aryl group;
n is 1, X is an N atom, R1 is a hydrogen atom and R2, R3, R2′, R3′, R3″ and R2″ are selected from a hydrogen atom, an alkyl group and an aryl group;
n is 1, X is a C atom, R1, R2, R2′, R2″, R3, R3′ and R3″ are hydrogen atoms and R1′ is a methyl (CH₃) group or a phenyl (C₆H₅) group;
n is 1, X is an N atom, R2, R2′, R2″, R3, R3′ and R3″ are hydrogen atoms and R1 is a phenyl (C₆H₅) group or a benzyl (CH₂C₆H₅) group
   for treating or preventing a cancer. Said cancer may be selected from skin cancer, colorectal cancer, lung cancer and breast cancer.
   A subject of the invention is also medicaments which comprise a compound of formula (I) according to the invention, or an addition salt of the latter with a pharmaceutically acceptable acid or else a hydrate or a solvate of said compound (I). These medicaments are of therapeutic use, in particular for the prevention and/or treatment of diseases involving an accumulation of AGEs or of ALEs, such as the pathological complications associated with diabetes (micro- and macroangiopathies, coronary and cerebrovascular atherosclerosis, retinopathy and nephropathy, healing problems), neurodegenerative diseases (Alzheimer's disease and Parkinson's disease), cataracts, osteoporosis, amyloidosis or skin aging.
   According to another aspect of the invention, a subject thereof is also a composition comprising at least one compound according to the invention. This may be pharmaceutical, cosmetic or agri-food compositions containing, as active ingredient, at least one compound according to the invention. These compositions contain an effective dose of a compound according to the invention, or of a pharmaceutically acceptable salt thereof, of one of the hydrates or of one of the solvates of said compound, and optionally one or more excipients which is (are) acceptable from a pharmaceutical, cosmetic or agri-food point of view. Said excipients are chosen, according to the pharmaceutical, cosmetic or agri-food form and the mode of administration desired, from the usual excipients which are known to those skilled in the art.
   In the compositions according to the present invention for oral, sublingual, subcutaneous, intramuscular, intravenous, transdermal, topical or rectal administration, the active ingredient of formula (I) above, or the optional salt, solvate or hydrate thereof, can be administered in unit administration form, as a mixture, for example, with conventional pharmaceutical, cosmetic or agri-food excipients, to animals and to human beings.
   The invention also relates to the use of a composition as defined above, in the cosmetics or agri-food field. According to one embodiment, it is a cosmetic use for treating or preventing skin aging.
   The active ingredient of formula (I) above, or the optional salt, solvate or hydrate thereof, in the pharmaceutical compositions according to the present invention for oral, sublingual, subcutaneous, intramuscular, intravenous, transdermal, topical or rectal use, can be administered in unit administration form, as a mixture, for example, with conventional pharmaceutical excipients, to animals and to human beings for the prophylaxis or treatment of the disorders or diseases mentioned above.
   According to another of its aspects, the present invention also relates to a method for treating the pathological conditions indicated above, which comprises the administration of a compound according to the invention, or of a pharmaceutically acceptable salt or of a hydrate of said compound.

### BRIEF DESCRIPTION OF THE FIGURES

FIGS. 1 and 2 represent curves of concentrations of glyoxal (GO) for FIG. 1 and of methylglyoxal (MGO) for FIG. 2, over time after the addition of a scavenger compound according to the invention (compounds 4c, 4e, 4h, 4k, 4l, 6, 11 and 13) or after the addition of a reference compound (aminoguanidine, D-Dap, D-Dap-L-Leu, 8-hydroxyquinoline).

FIG. 3 represents the measurement of the specific fluorescence making it possible to demonstrate the presence of advanced glycation end-products generated by the reaction of glucose and of some of its degradation products with proteins.

Other advantages may further appear to those skilled in the art on reading the examples below, illustrated by the appended figures, given by way of illustration.

### EXAMPLES

### I/ Synthesis of Compounds According to the Invention

### I.1. Synthesis of Compound 1

A suspension of (2R)-2,3-diaminopropanoic acid in monohydrochloride form (20.0 g; 142 mmol) in a mixture of dioxane (142 ml) and water (142 ml) is stirred at between 0 and 5° C. Triethylamine (59.0 ml; 426 mmol; 3 eq.) is then added to the medium which becomes homogeneous after a few minutes. Di-tert-butyl dicarbonate (62.0 g; 284 mmol; 2 eq.) is then added, in 4 portions, over the course of 30 min, taking care to keep the temperature below 10° C. The reaction mixture is kept stirring for 16 h at 20° C. The medium is then concentrated by 50%, under reduced pressure, before being taken up with diethyl ether (300 ml) and 1M hydrochloric acid (300 ml). After separation by settling out, the phases are separated and an extraction with diethyl ether (2×300 ml) is carried out on the aqueous phase. The combined organic phases are washed with brine (300 ml), dried over sodium sulfate, filtered and concentrated under vacuum. The residue obtained is dried for several hours under a high vacuum, and triturated, to give the expected compound 1 in the form of a white solid (42.4 g; 139 mmol; 98%).

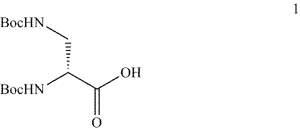 [SMILES: CC(C)(C)OC(=O)NC[C@@H](NC(=O)OC(C)(C)C)C(=O)O]

¹H NMR (CDCl₃, 300 MHz) δ (ppm): 9.40 (s, 1H); 6.29/5.80 (2s, 1H); 5.50/5.21 (2s, 1H); 4.33 (m, 1H); 3.54 (m, 2H), 1.44 (s, 18H).

¹³C NMR (CDCl₃, 75 MHz) δ (ppm): 173.4; 156.9; 156.3; 80.7; 80.4; 54.7; 42.2; 28.3.

### I.2. Synthesis of Compounds 3a-k

### General Method A—Coupling Reaction

1-Ethyl-3-(3-dimethylaminopropyl)carbodiimide hydrochloride (1.2 eq.) and then 1-hydroxybenzotriazole monohydrate (1.1 eq.) are successively added to a solution of compound 1 (1 eq.) in dichloromethane (10 ml/mmol), stirred at between 0 and 5° C. After stirring for 30 min at the same temperature, the amino derivative 2 (1.0 eq.), chosen according to the compound 3a, b, c, d, e, f, g, h, i, j or k targeted, is added and then the solution is kept stirring for 15 h while allowing the temperature to come back up to 20° C. The reaction mixture is then loaded with silica (1.5 g/mmol) before being concentrated under reduced pressure until a dry fine powder is obtained. A purification by silica gel chromatography is then carried out by depositing the powder obtained directly at the top of the column (eluent: cyclohexane/ethyl acetate, 70/30 to 40/60 or dichloromethane/methanol, 98/2 to 95/5). The expected compound 3a, b, c, d, e, f, g, h, i, j or k is obtained in the form of solid (76-98%).

### I.2.1. Compound 3a

The amino derivative 2 used to obtain the compound 3a is:

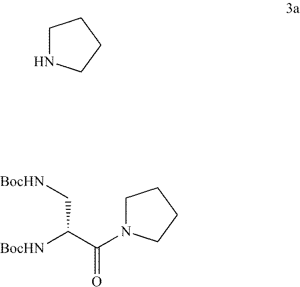 [SMILES: C1CCNC1 ; CC(C)(C)OC(=O)NC[C@@H](NC(=O)OC(C)(C)C)C(=O)N1CCCC1]

¹H NMR (CDCl₃, 300 MHz) δ (ppm): 5.57 (s, 1H); 5.15 (s, 1H); 4.54 (s, 1H); 3.75-3.28 (m, 5H); 3.24 (m, 1H); 2.02-1.78 (m, 4H); 1.41 (s, 18H).

¹³C NMR (CDCl₃, 75 MHz) δ (ppm): 168.5; 156.0; 155.5; 79.8; 79.4; 52.0; 46.4; 46.0; 42.6; 28.3; 26.0; 24.0.

### I.2.2. Compound 3b

The amino derivative 2 used to obtain the compound 3b is:

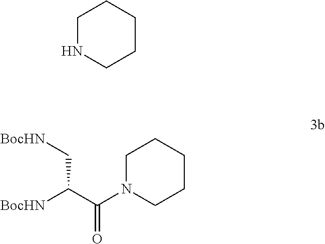 [SMILES: C1CCNCC1 ; CC(C)(C)OC(=O)NC[C@@H](NC(=O)OC(C)(C)C)C(=O)N1CCCCC1]

¹H NMR (CDCl₃, 300 MHz) δ (ppm): 5.60 (s, 1H); 5.05 (s, 1H); 4.72 (s, 1H); 3.70-3.32 (m, 5H); 3.22 (m, 1H); 1.68-1.49 (m, 6H); 1.42 (s, 18H).

¹³C NMR (CDCl₃, 75 MHz) δ (ppm): 168.0; 156.1; 155.4; 79.8; 79.4; 50.2; 46.6; 43.2; 28.3; 26.3; 25.4; 24.4.

### I.2.3. Compound 3c

The amino derivative 2 used to obtain the compound 3c is:

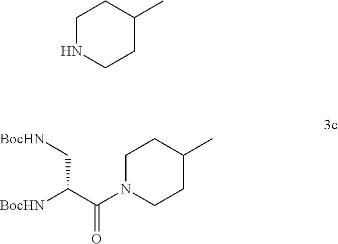

¹H NMR (CDCl₃, 300 MHz) δ (ppm): 5.63 (s, 1H); 5.04 (s, 1H); 4.72 (m, 1H); 4.48 (d, J=13.4 Hz, 1H); 3.93 (d, J=13.4 Hz, 1H); 3.37 (m, 1H); 3.20 (m, 1H); 3.04 (m, 1H); 2.58 (m, 1H); 1.64 (m, 3H); 1.42 (s, 18H); 1.30-0.97 (m, 2H); 0.93 (dd, ³J=6.2 Hz, ³J=6.2 Hz, 3H).

¹³C NMR (CDCl₃, 75 MHz) δ (ppm): 167.9; 155.9; 154.9; 79.7; 79.3; 53.3; 53.1; 45.9; 45.7; 43.5; 43.0; 42.7; 42.5; 34.5; 34.4; 33.6; 33.5; 31.0; 30.9; 38.3; 21.6; 21.5.

### I.2.4. Compound 3d

The amino derivative 2 used to obtain the compound 3d is:

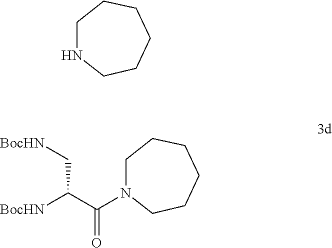 [SMILES: C1CCCNCC1 ; CC(C)(C)OC(=O)NC[C@@H](NC(=O)OC(C)(C)C)C(=O)N1CCCCCC1]

¹H NMR (CDCl₃, 300 MHz) δ (ppm): 5.55 (s, 1H); 5.14 (s, 1H); 4.72 (s, 1H); 3.94-3.05 (m, 6H); 2.01-1.27 (m, 26H).

¹³C NMR (CDCl₃, 75 MHz) δ (ppm): 169.9; 155.9; 155.3; 79.7; 79.3; 60.1; 47.7; 46.3; 43.2; 29.0; 28.3; 27.2; 27.1; 26.5.

### I.2.5. Compound 3e

The amino derivative 2 used to obtain the compound 3e is:

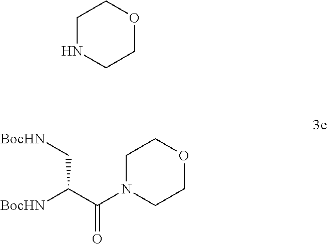 [SMILES: C1COCCN1 ; CC(C)(C)OC(=O)NC[C@@H](NC(=O)OC(C)(C)C)C(=O)N1CCOCC1]

¹H NMR (CDCl₃, 300 MHz) δ (ppm): 5.62 (d, ³J=7.9 Hz, 1H); 5.19 (s, 1H); 4.69 (m, 1H); 3.79-3.46 (m, 8H); 3.33 (m, 1H); 3.20 (m, 1H); 1.38 (s, 18H).

¹³C NMR (CDCl₃, 75 MHz) δ (ppm): 168.7; 155.9; 155.3; 79.8; 79.4; 66.5; 50.0; 45.9; 42.9; 42.4; 28.2.

### I.2.6. Compound 3f

The amino derivative 2 used to obtain the compound 3f is:

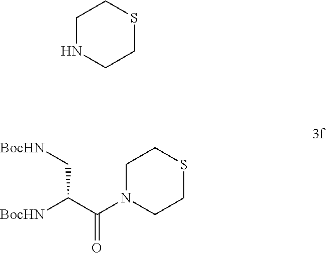 [SMILES: C1CSCCN1 ; CC(C)(C)OC(=O)NC[C@@H](NC(=O)OC(C)(C)C)C(=O)N1CCSCC1]

¹H NMR (CDCl₃, 300 MHz) δ (ppm): 5.58 (d, ³J=7.7 Hz, 1H); 5.08 (s, 1H); 4.71 (m, 1H); 3.91 (m, 2H); 3.77 (m, 2H); 3.33 (m, 1H); 3.21 (m, 1H); 2.62 (m, 4H); 1.40 (s, 18H).

¹³C NMR (CDCl₃, 75 MHz) δ (ppm): 168.8; 155.9; 155.3; 79.9; 79.5; 50.1; 48.2; 44.8; 43.0; 28.2; 27.8; 27.2.

### I.2.7. Compound 3q

The amino derivative 2 used to obtain the compound 3g is:

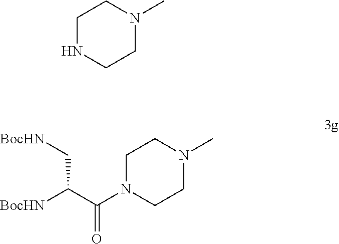

¹H NMR (CDCl₃, 300 MHz) δ (ppm): 5.59 (d, ³J=6.8 Hz, 1H); 5.13 (s, 1H); 4.70 (m, 1H); 3.57 (m, 4H); 3.34 (m, 1H); 3.13 (m, 1H); 2.38 (m, 4H); 2.25 (s, 3H); 1.38 (s, 18H).

¹³C NMR (CDCl₃, 75 MHz) δ (ppm): 168.4; 155.9; 155.3; 79.8; 79.4; 54.9; 54.4; 50.0; 45.8; 45.3; 43.1; 42.0; 28.3.

### I.2.8. Compound 3h

The amino derivative 2 used to obtain the compound 3h is:

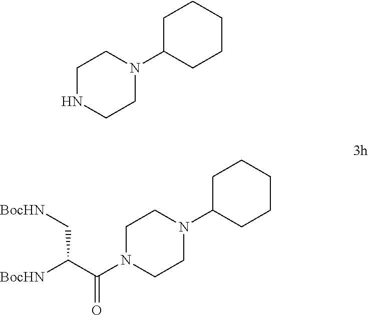 [SMILES: C1CCC(N2CCNCC2)CC1 ; CC(C)(C)OC(=O)NC[C@@H](NC(=O)OC(C)(C)C)C(=O)N1CCN(C2CCCCC2)CC1]

¹H NMR (CDCl₃, 300 MHz) δ (ppm): 5.58 (d, ³J=7.9 Hz, 1H); 5.07 (s, 1H); 4.72 (m, 1H); 3.66 (m, 4H); 3.37 (m, 1H); 3.15 (m, 1H); 2.51 (m, 4H); 2.24 (m, 1H); 1.77 (m, 4H); 1.58 (m, 2H); 1.39 (s, 18H); 1.17 (m, 4H).

¹³C NMR (CDCl₃, 75 MHz) δ (ppm): 168.1; 155.9; 155.3; 79.7; 79.3; 63.4; 50.0; 49.0; 48.5; 46.0; 43.2; 42.6; 28.8; 28.3; 26.1; 25.7.

### I.2.9. Compound 3i

The amino derivative 2 used to obtain the compound 3i is:

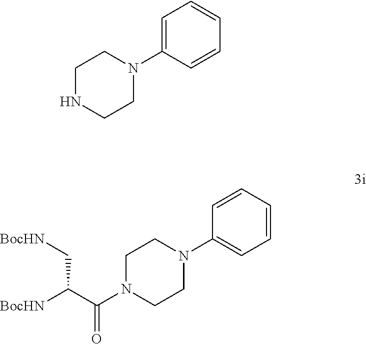 [SMILES: c1ccc(N2CCNCC2)cc1 ; CC(C)(C)OC(=O)NC[C@@H](NC(=O)OC(C)(C)C)C(=O)N1CCN(c2ccccc2)CC1]

¹H NMR (CDCl₃, 300 MHz) δ (ppm): 7.27 (m, 2H); 6.91 (m, 3H); 5.63 (d, ³J=7.4 Hz, 1H); 5.12 (s, 1H); 4.81 (m, 1H); 3.74 (m, 4H); 3.43 (m, 1H); 3.22 (m, 5H); 1.43 (s, 18H).

¹³C NMR (CDCl₃, 75 MHz) δ (ppm): 168.2; 155.6; 155.0; 150.3; 128.8; 120.1; 116.2; 79.5; 79.1; 49.8; 49.2; 48.8; 45.0; 42.7; 41.7; 27.9.

### I.2.10. Compound 3i

The amino derivative 2 used to obtain the compound 3j is:

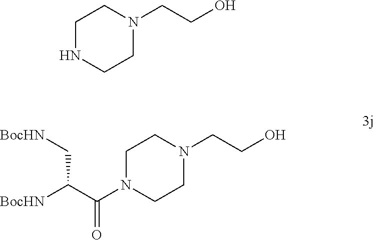 [SMILES: OCCN1CCNCC1 ; CC(C)(C)OC(=O)NC[C@@H](NC(=O)OC(C)(C)C)C(=O)N1CCN(CCO)CC1]

¹H NMR (CDCl₃, 300 MHz) δ (ppm): 5.70 (d, ³J=8.1 Hz, 1H); 5.24 (s, 1H); 4.66 (m, 1H); 3.66-3.40 (m, 6H); 3.26 (m, 1H); 3.14 (m, 1H); 2.96 (s, 1H); 2.58-2.28 (m, 6H); 1.33 (s, 18H).

¹³C NMR (CDCl₃, 75 MHz) δ (ppm): 168.4; 155.9; 155.2; 79.6; 79.2; 59.3; 57.8; 52.8; 52.3; 49.9; 45.2; 42.7; 41.9; 28.1.

### I.2.11. Compound 3k

The amino derivative 2 used to obtain the compound 3k is:

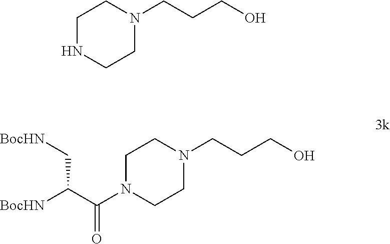 [SMILES: OCCCN1CCNCC1 ; CC(C)(C)OC(=O)NC[C@@H](NC(=O)OC(C)(C)C)C(=O)N1CCN(CCCO)CC1]

¹H NMR (CDCl₃, 300 MHz) δ (ppm): 5.62 (d, ³J=8.1 Hz, 1H); 5.05 (s, 1H); 4.71 (m, 1H); 3.76 (t, ³J=5.7 Hz, 2H); 3.69 (m, 4H); 3.36 (m, 1H); 3.23 (m, 1H); 2.75 (t, ³J=5.7 Hz, 2H); 2.65 (m, 5H); 1.79 (quint, ³J=5.7 Hz, 2H); 1.41 (s, 18H).

¹³C NMR (CDCl₃, 75 MHz) δ (ppm): 168.3; 156.1; 155.4; 80.0; 79.6; 62.9; 57.5; 52.8; 52.4; 50.3; 44.7; 43.0; 41.4; 28.3; 27.1.

### I.3. Synthesis of Compound 3l

1-Ethyl-3-(3-dimethylaminopropyl)carbodiimide hydrochloride (1.27 g; 6.6 mmol; 1.1 eq.) are added to a suspension of compound 1 (1.82 g; 6 mmol) and of 1-hydroxybenzotriazole monohydrate (1.01 g; 6.6 mmol; 1.1 eq.) in acetonitrile (60 ml), stirred at 0° C. After stirring for 15 min at 0° C., this solution is transferred into a dropping funnel and is then added (flow rate=1 ml/min) to a solution of piperazine (5.17 g; 60 mmol; 10 eq.) in acetonitrile (540 ml), at 0° C., with vigorous stirring. The reaction medium is kept stirring for 15 h while allowing the temperature to come back to 20° C. The mixture is then filtered through a sintered glass funnel before being concentrated under reduced pressure. The solid residue obtained is then purified, in three batches, by reverse-phase chromatography (Chromabond Flash RS40 C18ec column) according to the following conditions: elution gradient=10% B from 0 to 5 min, then 10% to 80% B from 5 to 35 min (with A=water and B=acetonitrile), flow rate=40 ml/min, detection=UV at 200 nm. The fractions collected at approximately 15 min are combined and lyophilized to give the expected compound 31 in the form of a white solid (1.68 g; 4.5 mmol; 75%).

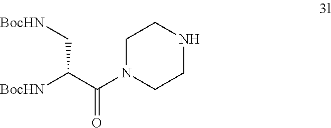 [SMILES: CC(C)(C)OC(=O)NC[C@@H](NC(=O)OC(C)(C)C)C(=O)N1CCNCC1]

¹H NMR (CDCl₃, 300 MHz) δ (ppm): 5.66 (s, 1H); 5.15 (s, 1H); 4.70 (s, 1H); 3.60-3.44 (m, 4H); 3.34 (m, 1H); 3.18 (m, 1H); 2.90-2.76 (m, 4H); 1.88 (s, 1H); 1.40 (s, 18H).

¹³C NMR (CDCl₃, 75 MHz) δ (ppm): 168.4; 155.9; 155.4; 79.8; 79.4; 50.0; 46.7; 46.1; 45.6; 43.2; 43.1; 28.2.

MS (ESI+): m/z=373 [M+H]; 395 [M+Na].

### I.4. Synthesis of Compound 3m

Potassium carbonate (207 mg; 1.5 mmol; 1 eq.) and then 1-bromobutane (165 μl; 1.5 mmol; 1 eq.) are successively added to a solution of the compound 3l (558 mg; 1.5 mmol) in acetonitrile (30 ml). The reaction medium is then kept stirring, at reflux, for 24 h, before being filtered through cotton wool and concentrated under reduced pressure. The residue obtained is purified by silica gel chromatography (eluent: dichloromethane/methanol, 95/5) to give the expected compound 3m in the form of a white solid (74%).

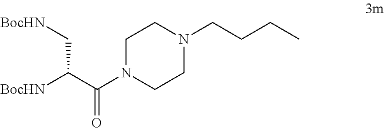 [SMILES: CCCCN1CCN(C(=O)[C@@H](CNC(=O)OC(C)(C)C)NC(=O)OC(C)(C)C)CC1]

¹H NMR (CDCl₃, 300 MHz) δ (ppm): 5.57 (d, ³J=7.2 Hz, 1H); 5.06 (s, 1H); 4.71 (m, 1H); 3.58 (m, 4H); 3.37 (m, 1H); 3.16 (m, 1H); 2.42 (m, 4H); 2.31 (m, 2H); 1.41 (s, 18H); 1.31 (m, 4H); 0.89 (t, ³J=7.2 Hz, 3H).

¹³C NMR (CDCl₃, 75 MHz) δ (ppm): 168.3; 155.9; 155.3; 79.8; 79.4; 58.1; 53.2; 52.7; 50.1; 45.5; 43.2; 42.1; 28.8; 28.3; 20.5; 13.9.

MS (ESI+): m/z=429 [M+H]; 451 [M+Na]; 492 [M+MeCN+Na].

### I.5. Synthesis of Compounds 5a-b

### I.5.1. Synthesis of Compound 5a

The general method A defined above was applied to compound 1, using piperazine (0.5 eq.) as amino derivative 2. The compound 5a is obtained in the form of a white solid (90%).

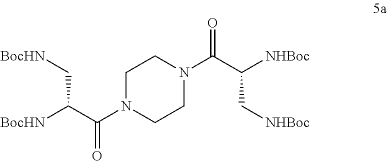 [SMILES: CC(C)(C)OC(=O)NC[C@@H](NC(=O)OC(C)(C)C)C(=O)N1CCN(C(=O)[C@@H](CNC(=O)OC(C)(C)C)NC(=O)OC(C)(C)C)CC1]

¹H NMR (CDCl₃, 300 MHz) δ (ppm): 5.58 (m, 2H); 5.04 (s, 2H); 4.71 (m, 2H); 3.86-3.47 (m, 8H); 3.35 (m, 4H); 1.42 (s, 36H).

¹³C NMR (CDCl₃, 75 MHz) δ (ppm): 168.8; 156.0; 155.3; 80.0; 79.7; 50.6; 50.3; 45.3; 45.0; 43.0; 42.1; 41.6; 28.3.

MS (ESI+): m/z=659 [M+H]; 681 [M+Na].

### I.5.2. Synthesis of Compound 5b

The general method A was applied to compound 1 using homopiperazine (0.5 eq.) as amino derivative 2. The compound 5b is obtained in the form of a white solid (84%).

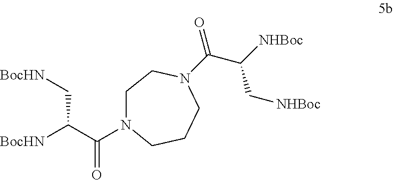 [SMILES: CC(C)(C)OC(=O)NC[C@@H](NC(=O)OC(C)(C)C)C(=O)N1CCCN(C(=O)[C@@H](CNC(=O)OC(C)(C)C)NC(=O)OC(C)(C)C)CC1]

¹H NMR (CDCl₃, 300 MHz) δ (ppm): 5.56 (m, 2H); 5.14 (s, 2H); 4.65 (m, 2H); 3.94-3.26 (m, 12H); 2.21-1.72 (m, 2H); 1.40 (s, 36H).

¹³C NMR (CDCl₃, 75 MHz) δ (ppm): 170.7; 170.5; 156.0; 155.4; 80.0; 79.6; 50.8; 50.5; 45.8; 48.1; 47.5; 47.3; 46.9; 46.4; 45.8; 44.9; 42.9; 42.7; 42.5; 28.3; 26.7.

MS (ESI+): m/z=673 [M+H]; 695 [M+Na].

### I.6. Synthesis of Compounds 4a-m and 6a-b

### General Method B—Deprotection Reaction

A 4M solution of hydrochloric acid in dioxane (2.5 ml/mmol; 10 eq.) is added to a solution of the compound 3a, b, c, d, e, f, g, h, i, j, k, l, m, 5a or 5b (1 eq.) in diethyl ether (1.25 ml/mmol). The reaction medium is then kept stirring for 12 h, during which time a precipitate gradually appears. The mixture is then concentrated under reduced pressure and the solid residue obtained is then dissolved in distilled water (5 ml/mmol). This aqueous solution is washed with diethyl ether (3×2.5 ml/mmol), filtered through a 0.45 μm membrane and lyophilized to give the corresponding expected compound, namely 4a, b, c, d, e, f, g, h, i, j, k, l, m, 6a or 6b, in hydrochloride form (85-98%).

### I.6.1. Synthesis of Compound 4a

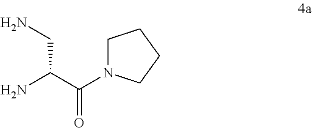 [SMILES: NC[C@@H](N)C(=O)N1CCCC1]

¹H NMR (D₂O, 300 MHz) δ (ppm): 4.72 (m, 1H); 3.77-3.42 (m, 6H); 1.99 (m, 4H).

¹³C NMR (D₂O, 75 MHz) δ (ppm): 163.3; 49.0; 46.7; 46.5; 38.0; 24.9; 23.1.

MS (ESI+): m/z=m/z=158 [M+H]; 180 [M+Na]; 199 [M+MeCN+H]; 221 [M+MeCN+Na]; 337 [2M+Na].

### I.6.2. Synthesis of Compound 4b

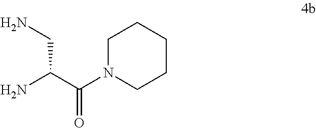 [SMILES: NC[C@@H](N)C(=O)N1CCCCC1]

¹H NMR (D₂O, 300 MHz) δ (ppm): 4.93 (m, 1H); 3.84-3.34 (m, 6H); 1.68 (s, 6H).

¹³C NMR (D₂O, 75 MHz) δ (ppm): 163.5; 48.2; 47.2; 44.4; 39.2; 25.9; 24.9; 23.4.

MS (ESI+): m/z=172 [M+H]; 194 [M+Na]; 213 [M+MeCN+H]; 235 [M+MeCN+Na].

### I.6.3. Synthesis of Compound 4c

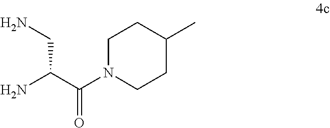

¹H NMR (D₂O, 300 MHz) δ (ppm): 4.95 (m, 1H); 4.32 (m, 1H); 3.87 (m, 1H); 3.55 (m, 2H); 3.29 (m, 1H); 2.85 (m, 1H); 1.79 (m, 3H); 1.19 (m, 2H); 0.96 (m, 3H).

¹³C NMR (D₂O, 75 MHz) δ (ppm): 163.1; 162.9; 47.7; 46.1; 45.7; 43.4; 42.9; 38.8; 38.5; 33.4; 33.1; 32.5; 32.1; 29.6; 29.2; 20.3; 19.9.

MS (ESI+): m/z=186 [M+H]; 208 [M+Na]; 393 [2M+Na].

### I.6.4. Synthesis of Compound 4d

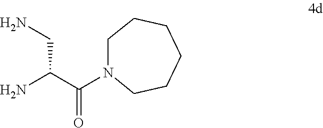 [SMILES: NC[C@@H](N)C(=O)N1CCCCCC1]

¹H NMR (D₂O, 300 MHz) δ (ppm): 4.88 (m, 1H); 3.83 (m, 1H); 3.69 (m, 1H); 3.59 (m, 2H); 3.50 (m, 1H); 3.30 (m, 1H); 1.89-1.68 (m, 4H); 1.61 (m, 4H).

¹³C NMR (D₂O, 75 MHz) δ (ppm): 164.5; 47.9; 47.6; 46.7; 38.7; 27.7; 26.1; 25.9; 25.3.

MS (ESI+): m/z=186 [M+H]; 208 [M+Na]; 393 [2M+Na].

### I.6.5. Synthesis of Compound 4e

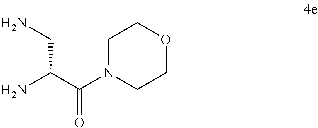 [SMILES: NC[C@@H](N)C(=O)N1CCOCC1]

¹H NMR (D₂O, 300 MHz) δ (ppm): 4.95 (m, 1H); 3.92-3.46 (m, 10H).

¹³C NMR (D₂O, 75 MHz) δ (ppm): 164.3; 66.0; 65.9; 48.0; 46.0; 43.0; 39.0.

MS (ESI+): m/z=174 [M+H]; 196 [M+Na]; 215 [M+MeCN+H]; 237 [M+MeCN+Na].

### I.6.6. Synthesis of Compound 4f

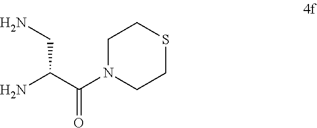 [SMILES: NC[C@@H](N)C(=O)N1CCSCC1]

¹H NMR (D₂O, 300 MHz) δ (ppm): 4.96 (dd, ³J=4.1 Hz, ³J=6.1 Hz, 1H); 4.21-4.09 (m, 1H); 4.05-3.94 (m, 1H); 3.89-3.80 (m, 1H); 3.72-3.61 (m, 1H); 3.57 (m, 2H); 2.96-2.66 (m, 4H).

¹³C NMR (D₂O, 75 MHz) δ (ppm): 163.7; 48.1; 47.7; 45.2; 38.5; 26.6; 25.9.

MS (ESI+): m/z=190 [M+H]; 212 [M+Na]; 401 [2M+Na].

### I.6.7. Synthesis of Compound 4q

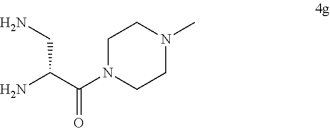

¹H NMR (D₂O, 300 MHz) δ (ppm): 5.01 (m, 1H); 4.62 (m, 1H); 4.27 (m, 1H); 3.85-3.53 (m, 5H); 3.41-3.14 (m, 3H); 3.00 (m, 3H).

¹³C NMR (D₂O, 75 MHz) δ (ppm): 164.0; 52.0; 51.9; 51.7; 47.7; 47.5; 42.6; 42.4; 42.3; 42.2; 39.4; 39.2; 38.7; 38.2.

MS (ESI+): m/z=187 [M+H]; 209 [M+Na]; 395 [2M+Na].

### I.6.8. Synthesis of Compound 4h

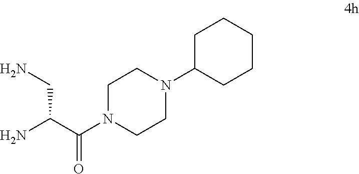 [SMILES: NC[C@@H](N)C(=O)N1CCN(C2CCCCC2)CC1]

¹H NMR (D₂O, 300 MHz) δ (ppm): 4.97 (m, 1H); 4.68 (m, 1H); 4.28 (m, 1H); 3.86-3.51 (m, 5H); 3.46-3.16 (m, 4H); 2.11 (m, 2H); 1.92 (m, 2H); 1.68 (m, 1H); 1.58-1.08 (m, 5H).

¹³C NMR (D₂O, 75 MHz) δ (ppm): 163.9; 65.9; 65.7; 47.6; 47.2; 46.8; 42.4; 39.5; 38.7; 38.3; 26.1; 24.0.

MS (ESI+): m/z=255 [M+H]; 277 [M+Na]; 531 [2M+Na].

### I.6.9. Synthesis of Compound 4i

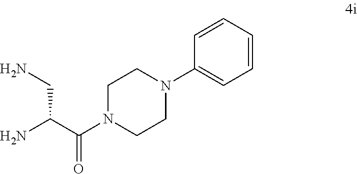 [SMILES: NC[C@@H](N)C(=O)N1CCN(c2ccccc2)CC1]

¹H NMR (D₂O, 300 MHz) δ (ppm): 7.60 (m, 5H); 5.07 (dd, ³J=3.8 Hz, ³J=6.2 Hz, 1H); 4.32-4.22 (m, 1H); 4.20-4.07 (m, 2H); 4.04-3.93 (m, 1H); 3.91-3.78 (m, 4H); 3.73-3.57 (m, 2H).

¹³C NMR (D₂O, 75 MHz) δ (ppm): 164.1; 140.7; 130.1; 129.5; 120.0; 53.3; 53.2; 47.6; 42.9; 40.0; 38.5.

MS (ESI+): m/z=249 [M+H]; 271 [M+Na]; 519 [2M+Na].

### I.6.10. Synthesis of Compound 41

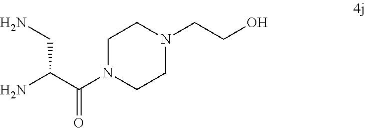 [SMILES: NC[C@@H](N)C(=O)N1CCN(CCO)CC1]

¹H NMR (D₂O, 300 MHz) δ (ppm): 5.01 (m, 1H); 4.24 (m, 1H); 3.98 (m, 2H); 3.90-3.72 (m, 3H); 3.69-3.52 (m, 4H); 3.51-3.18 (m, 4H).

¹³C NMR (D₂O, 75 MHz) δ (ppm): 164.0; 57.7; 54.3; 50.5; 47.6; 42.0; 39.0; 38.4.

MS (ESI+): m/z=217 [M+H]; 239 [M+Na]; 280 [M+MeCN+Na].

### I.6.11. Synthesis of Compound 4k

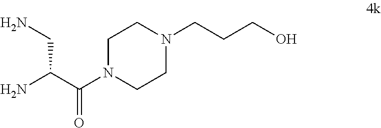 [SMILES: NC[C@@H](N)C(=O)N1CCN(CCCO)CC1]

¹H NMR (D₂O, 300 MHz) δ (ppm): 5.00 (m, 1H); 4.26 (m, 1H); 3.87-3.68 (m, 5H); 3.67-3.51 (m, 4H); 3.46-3.12 (m, 4H); 2.02 (m, 2H).

¹³C NMR (D₂O, 75 MHz) δ (ppm): 164.0; 58.0; 54.2; 50.5; 47.6; 42.1; 39.2; 38.4; 25.5.

MS (ESI+): m/z=231 [M+H]; 253 [M+Na]; 294 [M+MeCN+Na].

### I.6.12. Synthesis of Compound 4l

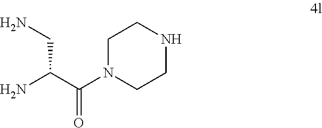 [SMILES: NC[C@@H](N)C(=O)N1CCNCC1]

¹H NMR (D₂O, 300 MHz) δ (ppm): 5.00 (dd, ³J=3.9 Hz, ³J=6.3 Hz, 1H); 4.17-3.28 (m, 10H).

¹³C NMR (D₂O, 75 MHz) δ (ppm): 164.7; 48.1; 42.8; 42.6; 42.5; 39.5; 39.0.

MS (ESI+): m/z=173 [M+H]; 195 [M+Na]; 214 [M+MeCN+H]; 236 [M+MeCN+Na]; 367 [2M+Na].

### I.6.13. Synthesis of Compound 4m

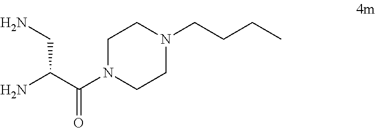 [SMILES: CCCCN1CCN(C(=O)[C@H](N)CN)CC1]

¹H NMR (D₂O, 300 MHz) δ (ppm): 5.00 (m, 1H); 4.61 (m, 1H); 4.27 (m, 1H); 3.86-3.51 (m, 5H); 3.40-3.11 (m, 5H); 1.76 (m, 2H); 1.41 (sext, ³J=7.3 Hz, 2H); 0.95 (t, ³J=7.3 Hz, 3H).

¹³C NMR (D₂O, 75 MHz) δ (ppm): 164.4; 164.0; 56.6; 56.2; 50.4; 50.0; 47.7; 47.5; 42.2; 42.1; 39.4; 39.1; 38.7; 38.3; 24.8; 18.7; 12.3.

MS (ESI+): m/z=229 [M+H]; 251 [M+Na].

### I.6.14. Synthesis of Compound 6a

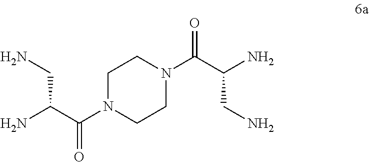 [SMILES: NC[C@@H](N)C(=O)N1CCN(C(=O)[C@H](N)CN)CC1]

¹H NMR (D₂O, 300 MHz) δ (ppm): 4.99 (m, 2H); 3.98 (m, 2H); 3.88-3.44 (m, 10H).

¹³C NMR (D₂O, 75 MHz) δ (ppm): 164.2; 164.0; 47.7; 47.6; 44.5; 43.9; 41.9; 41.4; 38.5; 38.4.

### I.6.15. Synthesis of Compound 6b

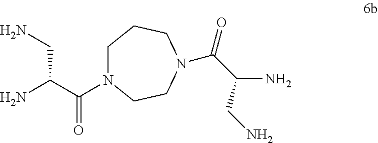 [SMILES: NC[C@@H](N)C(=O)N1CCCN(C(=O)[C@H](N)CN)CC1]

¹H NMR (D₂O, 300 MHz) δ (ppm): 4.91 (m, 2H); 4.16 (m, 2H); 3.91 (m, 2H); 3.68-3.45 (m, 6H); 3.42-3.09 (m, 2H); 2.18-1.82 (m, 2H).

¹³C NMR (D₂O, 75 MHz) δ (ppm): 165.0; 164.9; 48.3; 48.2; 48.1; 48.0; 47.8; 46.3; 45.9; 45.8; 45.2; 44.1; 38.7; 38.6; 38.4; 27.6; 27.0.

### I.6.16. High Resolution Mass Spectrometry HRMS (ES⁺) Values

Compound 4a. Calculated for [C₇H₁₅N₃O]H⁺ 158.1293. found 158.1301.
Compound 4b. Calculated for [C₈H₁₇N₃O]H⁺ 172.1450. found 172.1465.
Compound 4c. Calculated for [C₉H₁₉N₃O]H⁺ 186.1606. found 186.1619.
Compound 4d. Calculated for [C₉H₁₉N₃O]H⁺ 186.1606. found 186.1602.
Compound 4e. Calculated for [C₇H₁₅N₃O₂]H⁺ 174.1243. found 174.1259.
Compound 4f. Calculated for [C₇H₁₅N₃OS]H⁺ 190.1014. found 190.1023.
Compound 4g. Calculated for [C₈H₁₈N₄O]H⁺ 187.1559. found 187.1568.
Compound 4h. Calculated for [C₁₃H₂₆N₄O]H⁺ 255.2185. found 255.2184.
Compound 4i. Calculated for [C₁₃H₂₀N₄O]H⁺ 249.1715. found 249.1719.
Compound 4j. Calculated for [C₉H₂₀N₄O₂]H⁺ 217.1665. found 217.1657.
Compound 4k. Calculated for [C₁₀H₂₂N₄O₂]H⁺ 231.1821. found 231.1808.
Compound 4l. Calculated for [C₇H₁₆N₄O]H⁺ 173.1403. found 173.1416.
Compound 4m. Calculated for [C₁₁H₂₄N₄O]H⁺ 229.2028. found 229.2034.

### I.7. Synthesis of Compound 7

Hydrochloric acid at 37% (10 ml) is added, dropwise, with vigorous stirring, to a suspension of 8-hydroxyquinoline (2.9 g; 20 mmol) in formaldehyde (4 ml). The medium becomes homogeneous and bright yellow and heat is strongly given off during the addition. Hydrochloric acid gas is bubbled through the medium for 1 h and then the stirring is continued for 5 h. The temperature gradually decreases to approximately 25° C. and a fine yellow precipitate appears. The medium is then filtered and the residual yellow solid is washed with hydrochloric acid at 37% (4×5 ml), and then dried under a high vacuum. Compound 7 is obtained in the form of a bright yellow hygroscopic solid (3.15 g; 13.7 mmol; 68%) immediately stored under argon at 4° C.

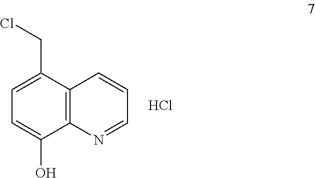 [SMILES: Oc1ccc(CCl)c2cccnc12 ; Cl]

¹H NMR (DMSO-d⁶, 300 MHz) δ (ppm): 9.24 (dd, ⁴J=1.2 Hz, ³J=8.7 Hz, 1H); 9.13 (dd, ⁴J=1.2 Hz, ³J=5.2 Hz, 1H); 8.13 (dd, ³J=5.2 Hz, ³J=8.7 Hz, 1H); 7.87 (d, ³J=8.1 Hz, 1H); 7.54 (d, ³J=8.1 Hz, 1H); 5.32 (s, 2H).

¹³C NMR (DMSO-d⁶, 75 MHz) δ (ppm): 149.5; 144.4; 142.9; 132.3; 129.6; 127.8; 124.6; 122.8; 115.2; 43.1.

### I.8. Synthesis of Compound 8

Molecular sieve 4 Å (6 g; preactivated for 3 h at 300° C.) and then the chlorinated compound 7 (1.38 g; 6 mmol; 1 eq.) are successively added to a solution, under an argon atmosphere, of potassium cyanide (1.98 g; 30 mmol; 5 eq.) in anhydrous dimethylformamide (30 ml). The stirring is maintained at 20° C. for 19 h, during which time the reaction mixture becomes green-yellowish in color. The medium is then filtered through cotton wool before being concentrated under reduced pressure. The residue obtained is taken up in water (20 ml) and this mixture is neutralized, carefully, by adding 1M hydrochloric acid. An extraction with dichloromethane (4×60 ml) is then carried out and the combined organic phases are then washed with brine (20 ml), dried over sodium sulfate, filtered and concentrated under reduced pressure. The pasty black solid obtained is washed with ether, triturated, and then dried under vacuum to enable the expected compound 8 to be obtained in the form of a light brown solid (974 mg; 5.3 mmol; 88%).

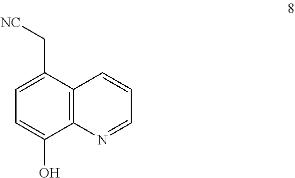 [SMILES: N#CCc1ccc(O)c2ncccc12]

¹H NMR (DMSO-d⁶, 300 MHz) δ (ppm): 9.97 (s, 1H); 8.92 (dd, ⁴J=1.5 Hz, ³J=4.1 Hz, 1H); 8.45 (dd, ⁴J=1.5 Hz, ³J=8.6 Hz, 1H); 7.67 (dd, ³J=4.1 Hz, ³J=8.6 Hz, 1H); 7.51 (d, ³J=7.9 Hz, 1H); 7.09 (d, ³J=7.9 Hz, 1H); 4.37 (s, 2H).

¹³C NMR (DMSO-d⁶, 75 MHz) δ (ppm): 153.4; 148.2; 138.7; 132.2; 127.9; 126.4; 122.1; 119.1; 117.0; 110.6; 19.3.

MS (ESI+): m/z=185 [M+H].

### I.9. Synthesis of Compound 9

A suspension of compound 8 (184 mg; 1 mmol) in hydrochloric acid at 37% (5 ml) is brought to reflux for 3 h. The medium is then concentrated under a high vacuum in order to provide the expected compound 9 in the form of a pale yellow solid (240 mg; 1 mmol; 100%).

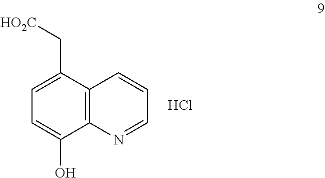 [SMILES: O=C(O)Cc1ccc(O)c2ncccc12 ; Cl]

¹H NMR (DMSO-d⁶, 300 MHz) δ (ppm): 9.11 (m, 2H); 8.07 (dd, ⁴J=5.2 Hz, ³J=8.6 Hz, 1H); 7.66 (s, 1H); 7.65 (d, ³J=7.9 Hz, 1H); 7.56 (d, ³J=7.9 Hz, 1H); 7.49 (s, 1H); 7.32 (s, 1H); 4.12 (s, 2H).

¹³C NMR (DMSO-d⁶, 75 MHz) δ (ppm): 172.2; 147.9; 144.0; 143.3; 131.6; 129.6; 128.7; 123.0; 122.0; 115.3; 36.8.

MS (ESI+): m/z=204 [M+H].

### I.10. Synthesis of Compound 10

Potassium carbonate (276 mg; 2 mmol; 2 eq.) and then the compound 31 (373 mg; 1 mmol; 1 eq.) are successively added to a suspension of compound 7 (230 mg; 1 mmol) in acetonitrile (10 ml). The reaction medium is kept vigorously stirring at 25° C. for 24 h before being filtered through cotton wool and concentrated under reduced pressure. The residue obtained is purified by reverse-phase chromatography (Chromabond Flash RS15 C18ec column) according to the following conditions: elution gradient=10% to 25% B from 0 to 40 min, 25% to 80% B from 40 to 45 min and then 80% B from 45 to 50 min (with A=water and B=acetonitrile), flow rate=15 ml/min, detection=UV at 200 nm. The fractions collected at approximately 41 min are combined and lyophilized to give the expected compound 10 in the form of a greenish solid (390 mg; 0.74 mmol; 74%).

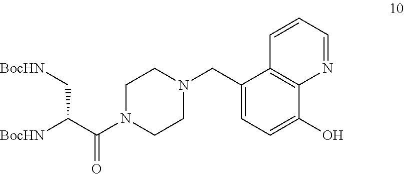 [SMILES: CC(C)(C)OC(=O)NC[C@@H](NC(=O)OC(C)(C)C)C(=O)N1CCN(Cc2ccc(O)c3ncccc23)CC1]

¹H NMR (CDCl₃, 300 MHz) δ (ppm): 8.79 (dd, ⁴J=1.5 Hz, ³J=4.2 Hz, 1H); 8.63 (dd, ⁴J=1.5 Hz, ³J=8.6 Hz, 1H); 7.46 (dd, ⁴J=4.2 Hz, ³J=8.6 Hz, 1H); 7.31 (d, ³J=7.8 Hz, 1H); 7.07 (d, ³J=7.8 Hz, 1H); 5.56 (d, ³J=7.7 Hz, 1H); 5.01 (m, 1H); 4.72 (m, 1H); 3.81 (s, 2H); 3.68-3.46 (m, 4H); 3.41 (m, 1H); 3.18 (m, 1H); 2.60-2.34 (m, 4H); 1.42 (s, 18H).

¹³C NMR (CDCl₃, 75 MHz) δ (ppm): 169.6; 156.0; 155.5; 152.0; 147.6; 138.8; 133.9; 129.1; 126.0; 123.7; 121.5; 108.6; 80.0; 79.2; 60.5; 52.9; 52.4; 50.3; 45.6; 43.3; 42.2; 28.3.

MS (ESI+): m/z=530 [M+H]; 552 [M+Na].

### I.11. Synthesis of Compound 11

The general method B was applied to compound 10 (390 mg; 0.74 mmol) previously synthesized, as compound 3 in said method defined above, to provide compound 11 corresponding to the deprotected compound 10 in the form of a yellow solid (270 mg; 0.57 mmol; 77%).

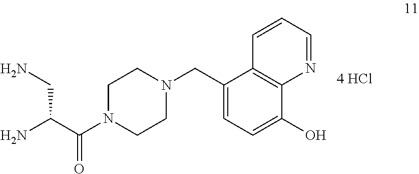 [SMILES: NC[C@@H](N)C(=O)N1CCN(Cc2ccc(O)c3ncccc23)CC1]

¹H NMR (D₂O, 300 MHz) δ (ppm): 9.39 (d, ³J=8.8 Hz, 1H); 9.13 (d, ³J=5.3 Hz, 1H); 8.22 (dd, ³J=8.8 Hz, ³J=5.3 Hz, 1H); 8.04 (d, ³J=8.1 Hz, 1H); 7.56 (d, ³J=8.1 Hz, 1H); 4.99 (m, 3H); 4.18-3.78 (m, 10H).

¹³C NMR (D₂O, 75 MHz) δ (ppm): 164.7; 149.9; 143.4; 142.8; 142.7; 136.6; 129.4; 123.0; 115.8; 115.6; 55.7; 50.9; 50.7; 48.1; 42.6; 39.7; 38.9.

MS (ESI+): m/z=330 [M+H]; 352 [M+Na]; 371 [M+MeCN+H].

### I.12. Synthesis of Compound 12

Triethylamine (404 μl; 3.6 mmol; 3.6 eq.), the compound 31 (372 mg; 1 mmol; 1 eq.), 1-ethyl-3-(3-dimethylaminopropyl)carbodiimide hydrochloride (230 mg; 1.2 mmol; 1.2 eq.) and then 1-hydroxybenzotriazole monohydrate (184 mg; 1.2 mmol; 1.2 eq.) are successively added to a suspension of compound 9 (240 mg; 1 mmol) in acetonitrile (20 ml). The reaction medium is kept stirring at 20° C. for 24 h, before being filtered through cotton wool, and then concentrated under reduced pressure. The reaction crude is purified by reverse-phase chromatography (Chromabond Flash RS40 C18ec column) according to the following conditions: elution gradient=10% B from 0 to 5 min and then 10% to 50% B from 5 to 35 min (with A=water and B=acetonitrile), flow rate=40 ml/min, detection=UV at 200 nm. The fractions collected at approximately 30 min are combined and lyophilized to give the expected compound 12 in the form of a beige solid (315 mg; 0.56 mmol; 56%).

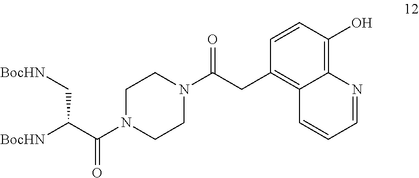 [SMILES: CC(C)(C)OC(=O)NC[C@@H](NC(=O)OC(C)(C)C)C(=O)N1CCN(C(=O)Cc2ccc(O)c3ncccc23)CC1]

¹H NMR (DMSO-d⁶, 300 MHz) δ (ppm): 8.79 (d, ³J=4.1 Hz, 1H); 8.38 (d, ³J=8.5 Hz, 1H); 7.48 (dd, ³J=4.1 Hz, ³J=8.5 Hz, 1H); 7.27 (d, ³J=7.8 Hz, 1H); 7.09 (d, ³J=7.8 Hz, 1H); 5.57 (d, ³J=7.3 Hz, 1H); 5.04 (m, 1H); 4.69 (m, 1H); 4.04 (m, 2H); 3.56 (m, 8H); 3.32 (m, 2H); 1.39 (m, 18H).

¹³C NMR (DMSO-d⁶, 75 MHz) δ (ppm): 169.5; 168.6; 155.9; 155.3; 151.7; 147.7; 138.6; 132.9; 128.2; 127.4; 121.9; 121.1; 109.2; 79.9; 79.5; 50.4; 45.9; 45.3; 42.9; 42.0; 41.3; 37.5; 28.3.

MS (ESI+): m/z=558 [M+H]; 580 [M+Na].

### I.13. Synthesis of Compound 13

The general method B was applied to compound 12 (315 mg; 0.56 mmol) previously synthesized, as compound 3 in said method defined above, to provide compound 13, corresponding to the deprotected compound 10, in the form of a yellowish solid (240 mg; 0.51 mmol; 92%).

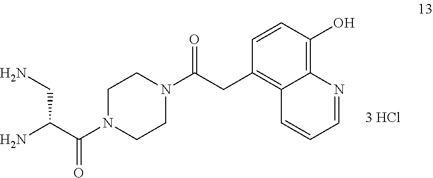 [SMILES: NC[C@@H](N)C(=O)N1CCN(C(=O)Cc2ccc(O)c3ncccc23)CC1]

¹H NMR (D₂O, 300 MHz) δ (ppm): 8.98 (m, 2H); 8.04 (dd, ³J=5.9 Hz, ³J=8.1 Hz, 1H); 7.55 (d, ³J=7.5 Hz, 1H); 7.35 (d, ³J=7.5 Hz, 1H); 5.01 (m, 1H); 4.36 (s, 2H); 4.10-3.50 (m, 10H).

¹³C NMR (D₂O, 75 MHz) δ (ppm): 171.1; 164.1; 145.9; 143.4; 141.9; 131.6; 128.8; 128.7; 123.1; 121.3; 115.5; 47.7; 44.7; 44.6; 44.4; 44.1; 42.1; 41.9; 41.2; 40.7; 38.5; 35.1.

MS (ESI+): m/z=358 [M+H]; 380 [M+Na].

HRMS (ES⁺): Compound 13. Calculated for [C₁₈H₂₃N₅O₃]H⁺ 358.1879. found 358.1877.

### II/ Demonstration of the Activity of Compounds According to the Invention as α-Oxoaldehyde Scavengers

### II.1. Principle

An aqueous solution of glucose (50 mM in phosphate buffer at 100 mM, pH=7.4) is incubated at 37° C. for 14 days in order to “naturally” generate glyoxal (GO) and methylglyoxal (MGO). The compound tested is introduced on the 7^{th} day (final concentration=100 μM). The GO and MGO concentrations of the medium are measured at regular intervals by LC-MS assaying, after derivatization with 2,3-diaminonaphthalene, in order to determine the effect of the compound tested.

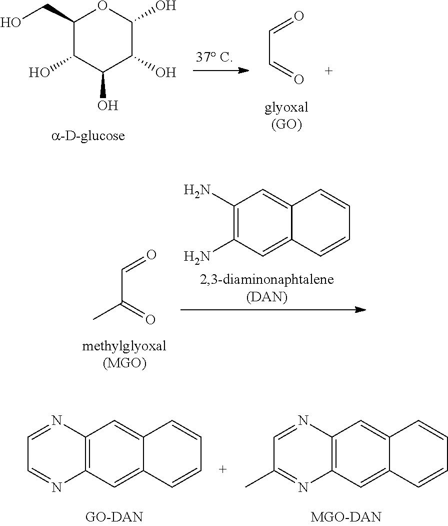

The results of the assays are compared with those obtained without the addition of scavenger compound (control) and with those obtained during the addition of a reference compound (aminoguanidine, D-Dap, D-Dap-L-Leu, 8-hydroxyquinoline).

### II.2. Experimental Protocol

A solution S₀ of glucose (50 mM) in phosphate buffer (100 mM, pH=7.4) is prepared and then filtered through a sterile 0.2 μm membrane into a sterile Falcon tube, under a vertical laminar flow filtering hood. 1000 μl of solution S₀ are then charged to several sterile capped Eppendorf tubes (1.5 ml) which are then stoppered and placed at 37° C., in the dark, for 14 days (“control” series). The rest of the solution S₀ is incubated in the same way, in a Falcon tube. The Eppendorf tubes are then removed, one by one, at regular intervals (approximately 24 h) and are immediately placed at −20° C. while awaiting analysis.

On D=7, an aqueous solution S₁ of the compound tested (5 mM) is prepared and then filtered through a sterile 0.2 μm membrane into a sterile Falcon tube, under a vertical laminar flow filtering hood. A mixture of the solution S₁ (140 μl) and of the remaining solution S₀, incubated for 7 days at 37° C. (6860 μl), is then prepared before being divided up between 6 sterile Eppendorf tubes (6×1000 μl) which are subsequently stoppered and placed at 37° C., in the dark, for 7 days (“test” series). These tubes are removed, one by one, at regular intervals (approximately 24 h) and are immediately placed at −20° C. while awaiting analysis.
On D=14, the tubes of the “control” and “test” series are thawed and are each treated with 100 μl of a solution of 2,3-diaminonaphthalene (10 mM) in order to derivatize the GO and the MGO, respectively in the form of GO-DAN and of MGO-DAN. After homogenization (vortex for 10 sec), the tubes are left to stand for 24 h, at 20° C., in the dark.
The GO-DAN and the MGO-DAN are then assayed in each sample by LC-MS (Shimadzu LCMS-2020), by external calibration carried out with standard solutions of GO-DAN and of MGO-DAN, according to the following conditions. Column: Shim-pack XR-ODS II (75×2 mm, 80 Å), temperature: 40° C., eluent: water/methanol (50/50)+0.1% of formic acid, flow rate: 300 μl/min, analysis time: 10 min, injection volume: 1 μl, detection: ESI+ in SIM mode (m/z=181.1 and 195.1) with the following parameters: interface voltage=4.5 kV, DL voltage=10 V, Q-array DC=0 V, Q-array RF=40 V (for m/z=181.1) or 10 V (for m/z=195.1). The GO-DAN and MGO-DAN compounds are respectively detected at the retention times of 3.9 min and 5.4 min.

### II.3. Results

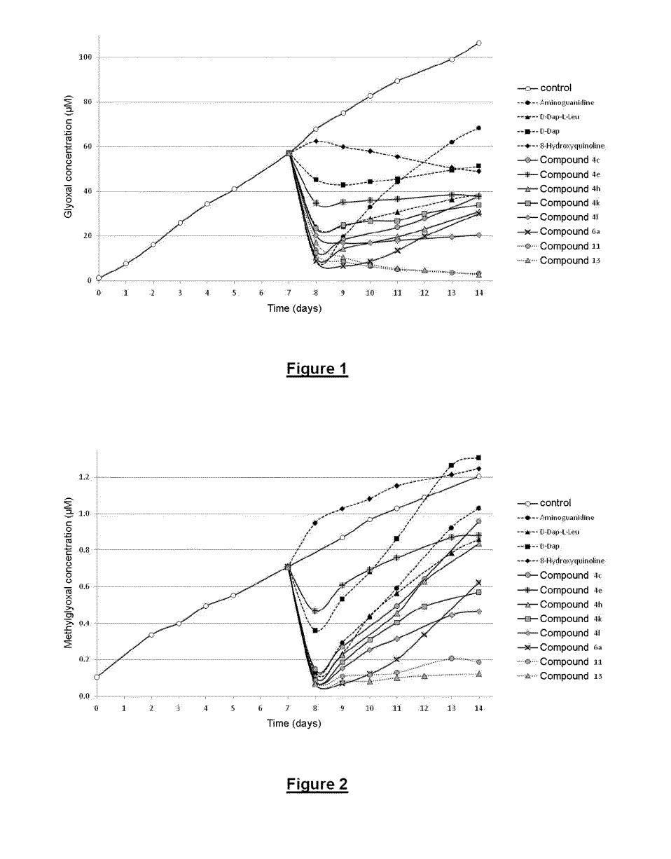

The glucose (50 mM), incubated at 37° C., slowly degrades and forms glyoxal (GO) and methylglyoxal (MGO). The concentrations of GO (FIG. 1) and of MGO (FIG. 2) increase linearly over time and reach, respectively, the values of 106 μM and 1.2 μM after 14 days.

The addition of compounds 4a-l or 6a makes it possible to significantly reduce the GO and MGO concentrations in less than 24 h. These compounds are found to be more reactive with respect to MGO than to GO and are on the whole better scavengers than the D-Dap or the D-Dap-L-Leu previously described. In particular, the compounds 4c, 4h and 6a show an activity close to or even greater than that of the reference compound, aminoguanidine.
The use of compounds 11 and 13 in this test also leads to very good results being obtained. Indeed, in addition to a good capacity for scavenging of GO and of MGO, these two compounds have, by virtue of the presence of the 8-hydroxyquinoline unit in their structure, the ability to inhibit the production of GO and of MGO from glucose. Extremely low levels of GO and of MGO are thus observed after 14 days: 3.0 μM (11) and 2.6 μM (13) of GO (compared with 68.4 μM for aminoguanidine) and 0.19 μM (11) and 0.12 μM (13) of MGO (compared with 1.20 μM for aminoguanidine).

### III/ Demonstration of the Activity of the Compounds According to the Invention as Inhibitors of AGE Formation

### III.1. Principle

The reaction of glucose and of some of its degradation products with proteins generates advanced glycation end-products (AGEs) among which certain species exhibit a specific fluorescence which can be used to demonstrate their presence.

Human albumin at physiological concentration (50 g/l) is incubated with glucose (500 mM in phosphate buffer at 100 mM, pH=7.4) at 37° C. for 20 days in the presence or absence of the compounds tested (50 mM). A measurement of the fluorescence (reading at 440 nm after excitation at 370 nm) is then carried out on each sample. The results are expressed in the form of a fluorescence read (sample)/maximum fluorescence observed (control without scavenger compound, D=20) ratio.

### III.2. Experimental Protocol

A solution of human albumin (50 g/l) and of glucose (500 mM) in phosphate buffer (100 mM, pH=7.4) is prepared. 1000 μl of the previous solution are then immediately charged to tubes, each containing one of the compounds tested (50 μmol). The resulting solutions are homogenized (vortex for 10 sec) and then immediately filtered through a sterile 0.2 μm membrane into sterile capped Eppendorf tubes (1.5 ml), under a vertical laminar flow filtering hood. The tubes are then stoppered and placed at 37° C., in the dark, for 20 days. After a return to ambient temperature (22° C.), a measurement of the fluorescence (λₑₓ=370 nm, λₑₘ=440 nm) is carried out on 200 μl of each sample.

### III.3. Results

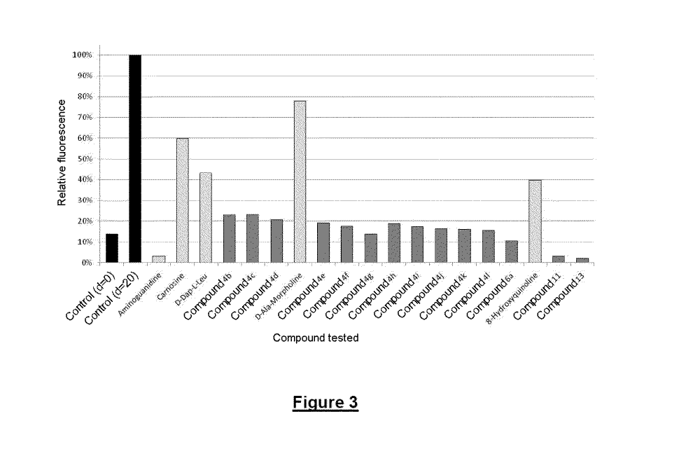

The control sample of albumin incubated alone in the glucose solution experiences a multiplication of its fluorescence (and therefore of the amount of AG Es present) by a factor of approximately 7 after 20 days (relative fluorescence: 14% on D=0 and 100% on D=20) (FIG. 3).

The presence, in the medium, of known compounds which inhibit AGE formation results in an expected decrease of the fluorescence. Among these reference compounds, aminoguanidine is found to be the most effective (relative fluo.: 3%), far ahead of carnosine (60%) and D-Dap-L-Leu (43%).
The addition of the compounds 4a-l and 6a also makes it possible to maintain low AGE levels (relative fluo.: 11-23%), close to the initial level of the control (14% on D=0). The very large difference in activity between the compound 4e (relative fluo.: 19%) and D-Ala-Morpholine (78%), a derivative of analogous structure which has only one amine function, can be noted, this being evidence of the involvement of the 1,2-diamine unit in the anti-AGE activity of the compounds described here.
Compounds 11 and 13, derived from 8-hydroxyquinoline, are found to be the most effective of the series of molecules tested since they produce the lowest relative fluorescence values observed (respectively 3% and 2%), well below that obtained for 8-hydroxyquinoline (40%) and of the same order of magnitude as that observed for aminoguanidine (3%).

1. A compound of formula (I) below, in free form or in the form of a salt of at least one inorganic or organic acid, 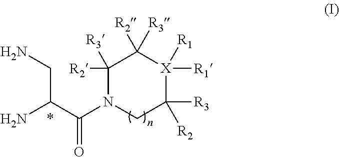
in which:
R2, R2′ and R2″ are selected from a hydrogen atom, an alkyl group, an aralkyl group and an aryl group;
R3, R3′ and R3″ are selected from a hydrogen atom, an alkyl group, an aralkyl group and an aryl group;
n is an integer equal to 0, 1 or 2;
X is an atom selected from O, S, C and N atoms;
provided that, when X is an O or S atom, then R1 and R1′ are absent; and
provided that, when X is a C atom, then
(i) R1 and R1′, independently of one another, are selected from a hydrogen atom, an alkyl group and an aryl group, or
(ii) R1 is selected from a hydrogen atom, an alkyl group and an aryl group, and R1′ is connected to R2, R2′, R2″, R3, R3′ or R3″ with a methylene (—CH₂—), oxo (—O—), thio (—S—) or imino (—NH—) moiety so as to form a ring, or
(iii) R1 and R1′, independently of one another, are connected to R2, R2′, R2″, R3, R3′ or R3″ with a methylene (—CH₂—), oxo (—O—), thio (—S—) or imino (—NH—) moiety so as to form a ring, or
provided that, when X is an N atom, then (i) R1′ is absent and (ii) R1 is selected from a hydrogen atom, an alkyl group, a hydroxyalkyl group, an aralkyl group, an aryl group,
a group 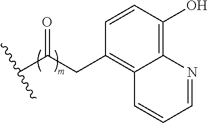
and a group 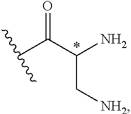 [SMILES: *C(=O)C(N)CN]
with m an integer selected from 0 and 1, or R1 is connected to R2, R2′, R2″, R3, R3′ or R3″ with a methylene (—CH₂—), oxo (—O—), thio (—S—) or imino (—NH—) moiety so as to form a ring,
and wherein:
“*” denotes that the corresponding asymmetric carbon atom is of R or S stereochemistry;
with the proviso that the following compounds are excluded:
n is 0, X is a C atom and R1, R1′, R2, R2′, R2″, R3, R3′ and R3″ are hydrogen atoms;
n is 1 and X is an O or S atom;
n is 1, X is a C atom and R1 and R1′ are hydrogen atoms;
n is 1, X is an N atom and R1 is a hydrogen atom;
n is 1, X is a C atom, R1, R2, R2′, R2″, R3, R3′ and R3″ are hydrogen atoms and R1′ is a methyl (—CH₃) group or a phenyl (—C₆H₅) group;
n is 1, X is an N atom, R2, R2′, R2″, R3, R3′ and R3″ are hydrogen atoms and R1 is a phenyl (C₆H₅) group or a benzyl (CH₂C₆H₅) group.

2. Compound as claimed in claim 1, of formula (Ia) below: 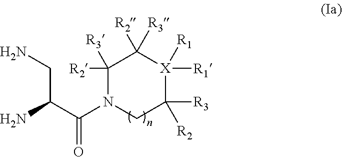
or of formula (Ib) below:

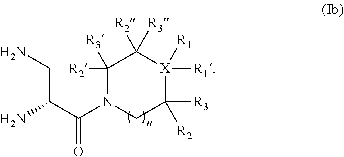

3. Compound as claimed in claim 2, of formula (Ia) below: 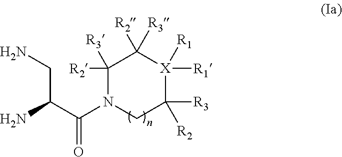
wherein R1 is selected from the following moiety:

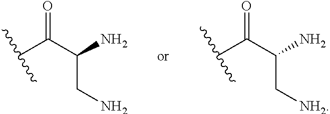 [SMILES: *C(=O)[C@@H](N)CN ; *C(=O)[C@H](N)CN]

4. Compound as claimed in claim 2, of formula (Ib) below: 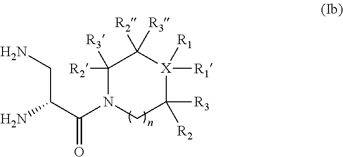
wherein R1 is selected from the following moiety:

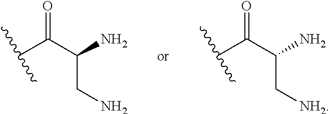 [SMILES: *C(=O)[C@@H](N)CN ; *C(=O)[C@H](N)CN]

5. Compound as claimed in any claim 1, wherein n is 0 and X is a nitrogen atom.

6. Compound as claimed in claim 1, wherein:
R2′, R2″, R3′ and R3″ are other than a hydrogen atom; or
R2″ is a hydrogen atom and R2′, R3′ and R3″ are other than a hydrogen atom; or
R2′ is a hydrogen atom and R2″, R3″ and R3′ are other than a hydrogen atom; or
R2′ and R3′ are hydrogen atoms and R2″ and R3″ are other than a hydrogen atom; or
R2″ and R3″ are hydrogen atoms and R2′ and R3′ are other than a hydrogen atom; or
R2′ and R2″ are hydrogen atoms and R3′ and R3″ are other than a hydrogen atom; or
R2′, R2″, R3′ and R3″ are hydrogen atoms.

7. Compound as claimed in claim 1, wherein the organic or inorganic acid is selected from hydrochloric, hydrobromic, hydroiodic, sulfuric, phosphoric, tartaric, lactic, acetic, adipic, alginic, aspartic, benzoic, benzenesulfonic, bisulfic, butyric, citric, camphoric, camphorsulfonic, gluconic, dodecylsulfonic, ethanesulfonic, fumaric, glucoheptanoic, heptanoic, hexanoic, 2-hydroxyethanesulfonic, maleic, methanesulfonic, 2-naphthalenesulfonic, nicotinic, oxalic, palmoic, palmitic, pectinic, 3-phenylpropionic, picric, pivalic, propionic, succinic and undecanoic acids.

8. Compound as claimed in claim 1, selected from:
4d: (2R)-2,3-diamino-1-(azepan-1-yl)propan-1-one;
4g: (2R)-2,3-diamino-1-(4-methylpiperazin-1-yl)propan-1-one;
4h: (2R)-2,3-diamino-1-(4-cyclohexylpiperazin-1-yl)propan-1-one;
4j: (2R)-2,3-diamino-1-[4-(2-hydroxyethyl)piperazin-1-yl]propan-1-one;
4k: (2R)-2,3-diamino-1-[4-(3-hydroxypropyl)piperazin-1-yl]propan-1-one;
4m: (2R)-2,3-diamino-1-(4-butylpiperazin-1-yl)propan-1-one;
6a: (2R,2′R)-1,1′-(piperazine-1,4-diyl)bis(2,3-diaminopropan-1-one);
6b: (2R,2′R)-1,1′-(1,4-diazepane-1,4-diyl)bis(2,3-diaminopropan-1-one);
11: (2R)-2,3-diamino-1-{4-[(8-hydroxyquinolin-5-yl)methyl]piperazin-1-yl}propan-1-one; and
13: (2R)-2,3-diamino-1-{4-[2-(8-hydroxyquinolin-5-yl)acetyl]piperazin-1-yl}propan-1-one.

9. Use of a compound as claimed in claim 1, for scavenging an alpha-oxoaldehyde or an alpha, beta-unsaturated aldehyde.

10. Use as claimed in claim 9, wherein the alpha-oxoaldehyde results from glucose degradation or wherein the alpha, beta-unsaturated aldehyde results from the oxidative degradation of a fatty acid.

11. Use as claimed in claim 10, wherein the alpha-oxoaldehyde resulting from glucose degradation is selected from glyoxal, methylglyoxal and 3-deoxyglucosone or wherein the alpha, beta-unsaturated aldehyde resulting from the oxidative degradation of a fatty acid is selected from acrolein, malondialdehyde and 4-hydroxynonenal.

12. Compound as claimed in any claim 1, for use as an alpha-oxoaldehyde scavenger or as an alpha, beta-unsaturated aldehyde scavenger.

13. Compound as claimed in claim 1, for use as a medicament.

14. Compound as claimed in any claim 1 or the compound of formula (I) 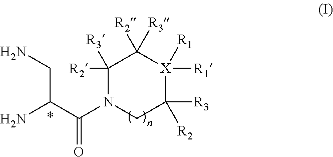
in which:
n is 0, X is a C atom and R1, R1′, R2, R2′, R2″, R3, R3′ and R3″ are hydrogen atoms;
n is 1, X is an O or S atom, R1 and R1′ are absent and R2, R3, R2′, R3′, R3″ and R2″ are selected from a hydrogen atom, an alkyl group and an aryl group;
n is 1, X is a C atom, R1 and R1′ are hydrogen atoms and R2, R3, R2′, R3′, R3″ and R2″ are selected from a hydrogen atom, an alkyl group and an aryl group;
n is 1, X is an N atom, R1 is a hydrogen atom and R2, R3, R2′, R3′, R3″ and R2″ are selected from a hydrogen atom, an alkyl group and an aryl group;
n is 1, X is a C atom, R1, R2, R2′, R2″, R3, R3′ and R3″ are hydrogen atoms and R1′ is a methyl (CH₃) group or a phenyl (C₆H₅) group;
n is 1, X is an N atom, R2, R2′, R2″, R3, R3′ and R3″ are hydrogen atoms and R1 is a phenyl (C₆H₅) group or a benzyl (CH₂C₆H₅) group,
for treating or preventing a neurodegenerative pathological condition.

15. Compound as claimed in claim 14, wherein the neurodegenerative pathological condition is selected from Alzheimer's disease and Parkinson's disease.

16. Compound as claimed in claim 1 or the compound of formula (I) 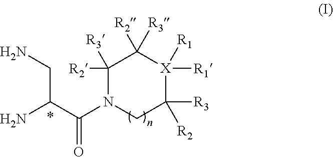
in which:
n is 0, X is a C atom and R1, R1′, R2, R2′, R2″, R3, R3′ and R3″ are hydrogen atoms;
n is 1, X is a C atom, R1, R2, R2′, R2″, R3, R3′ and R3″ are hydrogen atoms and R1′ is a methyl (CH₃) group or a phenyl (C₆H₅) group;
n is 1, X is an N atom, R2, R2′, R2″, R3, R3′ and R3″ are hydrogen atoms, R1′ is absent and R1 is a phenyl (C₆H₅) group or a benzyl (CH₂C₆H₅) group,
for treating or preventing conditions associated with diabetes.

17. Compound as claimed in claim 16, wherein the conditions associated with diabetes are selected from atherosclerosis, retinopathy, nephropathy, neuropathy, micro- and macroangiopathies, cataracts, amyloidosis, rheumatic disorders and varicose and arterial ulcers.

18. Compound as claimed in claim 1 or the compound of formula (I) 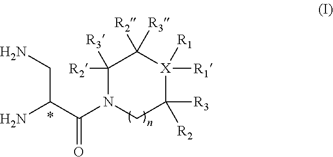
in which:
n is 0, X is a C atom and R1, R1′, R2, R2′, R2″, R3, R3′ and R3″ are hydrogen atoms;
n is 1, X is an O or S atom, R1 and R1′ are absent and R2, R3, R2′, R3′, R3″ and R2″ are selected from a hydrogen atom, an alkyl group and an aryl group;
n is 1, X is a C atom, R1 and R1′ are hydrogen atoms and R2, R3, R2′, R3′, R3″ and R2″ are selected from a hydrogen atom, an alkyl group and an aryl group;
n is 1, X is an N atom, R1 is a hydrogen atom and R2, R3, R2′, R3′, R3″ and R2″ are selected from a hydrogen atom, an alkyl group and an aryl group;
n is 1, X is a C atom, R1, R2, R2′, R2″, R3, R3′ and R3″ are hydrogen atoms and R1′ is a methyl (CH₃) group or a phenyl (C₆H₅) group;
n is 1, X is an N atom, R2, R2′, R2″, R3, R3′ and R3″ are hydrogen atoms and R1 is a phenyl (C₆H₅) group or a benzyl (CH₂C₆H₅) group
for treating or preventing a cancer.

19. Compound as claimed in claim 18, wherein the cancer is selected from skin cancer, colorectal cancer, lung cancer and breast cancer.

20. A composition comprising at least one compound as claimed in claim 1.

21. Use of a composition as claimed in claim 20, in the cosmetics or agri-food field.

22. The cosmetic use as claimed in claim 21, for treating or preventing skin aging.
